(19) United States
(12) Patent Application Publication
(10) Pub. No.: US 2023/0049974 A1
(43) Pub. Date: Feb. 16, 2023

(54) FINGOLIMOD EXTENDED RELEASE INJECTABLE SUSPENSION

(71) Applicant: SHILPA MEDICARE LIMITED, RAICHUR (IN)

(72) Inventors: KIRAN KRISHNAPPA JADHAV, Bengaluru (IN); SREENIVASA REDDY, Bengaluru (IN)

(73) Assignee: SHILPA MEDICARE LTD, RAICHUR (IN)

(21) Appl. No.: 17/788,297

(22) PCT Filed: Jan. 4, 2021
(86) PCT No.: PCT/IB2021/050008

§ 371 Date: Jun. 23, 2022

(30) Foreign Application Priority Data
Jan. 6, 2020 (IN) 202041000435

(51) Int. Cl. A61K 31/137; A61K 9/00; A61K 47/38

(52) U.S. Cl. CPC A61K 31/137; A61K 9/0019; A61K 47/38

(57) ABSTRACT

The present invention relates to an injectable composition for extended release of fingolimod comprising a suspension of at least about 0.5 mg/ml of fingolimod, wherein fingolimod release is for at least 7 days and the process for preparation thereof.

### FIELD OF THE INVENTION

The present invention relates to the fingolimod extended release suspension injection indicated for treatment of relapsing forms of multiple sclerosis (MS) to reduce the frequency of clinical exacerbations and to delay the accumulation of physical disability.

### BACKGROUND OF THE INVENTION

Fingolimod (FTY 720), is an immunomodulating compound and is structurally represented as

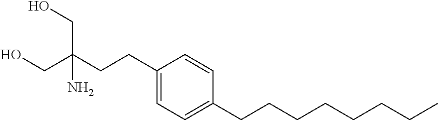 [SMILES: CCCCCCCCc1ccc(CCC(N)(CO)CO)cc1]

It is a structural analogue of sphingosine and its pharmaceutical activity is associated with its modulating activity towards sphingosine-1-phosphate (SIP) receptors. Fingolimod is metabolized by sphingosine kinase to the active metabolite fingolimod phosphate.

Fingolimod is approved as Gilenya™, which is a hard-shell capsule filled by a powder comprising 0.56 mg of micronized fingolimod hydrochloride corresponding to 0.5 mg of fingolimod per capsule, for once daily administration for the treatment of relapsing remitting multiple sclerosis. Gilenya™ has very fast dissolution profile, which provide dissolution of about 99% in 30 minutes in 500 ml media constituted by 0.1 N HCL+0.2% SLS, at 100 rpm (as specified by Office of generic drugs or OGD). Gilenya™ capsule comprises mannitol as diluent, prepared by direct blending method and capsule additionally comprises small amount of magnesium stearate as a lubricant.

However, in addition to side effects common to immunomodulatory therapy, FTY720 was reported to cause cardiovascular complications, macular oedema, and brain inflammation, which may be due to FTY720 interacting with more than one SIP-receptor subtypes. Martin R., Nature, 464, 360-362 (2010). Accordingly, there remains a need for the development of more effective FTY720 derivatives.

However, there exists a need to develop the fingolimod extended release suspension injection indicated for treatment of relapsing forms of multiple sclerosis (MS) to reduce the frequency of clinical exacerbations and to delay the accumulation of physical disability.

### OBJECTS OF THE INVENTION

In one object the present invention provides herein, fingolimod extended release suspension injection compositions or formulations consisting essentially of a therapeutically effective amount of fingolimod and an excipient that facilitates intramuscular administration, and methods of use thereof for treating patients with relapsing forms of multiple sclerosis (MS) to reduce the frequency of clinical exacerbations and to delay the accumulation of physical disability.

In another object, the present invention further provides the fingolimod extended release suspension injection with sustained release of the drug (fingolimod) from the site of injection for about 7 days, 14 days, 21 days, 28 days, 35 days, 42 days, 49 days, 56 days, 63 days, 70 days, 77 days, 84 days, 91 days and 98 days.

In a further object, the present invention provides the fingolimod extended release suspension injection with sustained release of drug (fingolimod) from the site of injection for 7 days, 14 days, 21 days, 28 days, 35 days, 42 days, 49 days, 56 days, 63 days, 70 days, 77 days, 84 days, 91 days and 98 days, wherein the fingolimod release from the injection site is about 0.05 mg/day to about 1.5 mg/day, preferably from about 0.1 mg/day to about 1.3 mg/day and more preferably from about 0.15 mg/day to about 1.0 mg/day.

In another object, the present invention provides method of intramuscular administration of fingolimod extended release suspension injection, wherein the mean residence time (MRT) of the fingolimod extended release suspension by intramuscular administration is at least twice the mean residence time (MRT) of fingolimod injection by intravenous administration.

In yet another object, the present invention provides the fingolimod extended release suspension injection composition comprising fingolimod, suspending agent and purified water,

In most preferred object, the present invention provides the fingolimod extended release suspension injection composition comprising fingolimod, suspending agent, tonicity adjusting agent and purified water.

### SUMMARY OF THE INJECTION

The present invention relates, in part, to the discovery that a pharmaceutical composition comprising fingolimod administered as bolus injection by intramuscular administration resulted in an extended release profile of fingolimod. The extended release injection of fingolimod results in patient compliance and maximizing the pharmacological profile of fingolimod.

In embodiment of the invention, the present invention provides an injectable composition for the extended release of fingolimod by intramuscular administration wherein fingolimod is present in the serum of the mammal for at least about 7 days, 14 days, 21 days, 28 days, 35 days, 42 days, 49 days, 56 days, 63 days, 70 days, 77 days, 84 days, 91 days and 98 days. Most preferably, fingolimod is present in the serum of the mammal for at least about 7 days, 14 days, 21 days and 28 days. In a preferred embodiment, the composition comprises a suspension of fingolimod and a suspending agent. The fingolimod drug substance can comprise, consist essentially of or consist of fingolimod (in a crystalline, non-crystalline or amorphous form), a fingolimod salt (fingolimod HCl, fingolimod phosphate), a fingolimod solvate (including hydrates) or other fingolimod polymorphs. The fingolimod or fingolimod drug substance, can be added in a specified size. For example, the fingolimod or fingolimod drug substance can be added after being micronized to D90 of less than about 100 microns, preferably less than about 75 microns, more preferably less than about 60 microns and most preferably between about 5 microns and 50 microns, as determined by master sizer 3000 particle analyzer.

In one embodiment, the extended release suspension composition comprising fingolimod and a suspending agent is injected to a mammal comprising of at least about 0.5 mg of fingolimod, such as at least about 1 mg to about 5 mg, or as much as about 7 mg to 10 mg, e.g less than 20 mg. The fingolimod can be present in an amount of at least about 0.5 mg/mL, preferably at least about 1 mg/mL to about 10 mg/mL, more preferably 1.5 mg/mL, 2 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL and 10.0 mg/mL.

In other embodiment the present invention relates to an injectable composition for extended release of fingolimod comprising a suspension of at least about 0.5 mg/mL of fingolimod. The invention also relates to an extended release composition of fingolimod comprising at least about 0.5 mg/mL of fingolimod and a suspending agent and to the compositions useful in such methods.

DEFINITION OF SELECTED TERMS

In describing and claiming the present invention, the following terminologies will be used in accordance with the definitions set out below.

The term “injection” is the act of putting a liquid, especially a drug, into a person's body using a needle (usually a hypodermic needle) and a syringe. Injection is a technique for delivering drugs by parenteral administration, that is, administration via a route other than through the digestive tract. Parenteral injection includes subcutaneous, intramuscular, intravenous, intraperitoneal, intracardiac, intraarticular and intracavernous injection.

The term “individual”, “subject” or “patient” refers to a warm blooded animal, including but not limited to humans, such as a mammal which is afflicted with a particular disease state.

The term of “intramuscular injection” is the injection of a substance directly into muscle.

The term mean residence time (MRT) as used herein refers to an average time a drug molecule spends in the body. MRT is defined as the average time intact drug molecule transit or reside in the body. For a population of drug molecules individual molecules spend different times within the body. Following the principles of statistical probability, specific drug molecule may be eliminated quickly, whereas others may remain in the body much longer. Consequently, a distribution of transit time can be characterized by a mean value. In other words, elimination of a drug can be thought of as random process. Residence time reflects how long a particular drug molecule remains or resides in the body. The MRT reflect the overall behavior of a large number of molecules.

The term “multiple sclerosis (MS)” as used herein refers to as a frequent and disabling neurological disease characterized by multifocal destruction of central nervous system myelin.

The term “therapeutically effective amount” is further meant to define an amount resulting in improvement of any parameter or clinical symptoms. The actual dose may vary with each patient and does not necessarily indicate a total elimination of all disease symptoms.

### BRIEF DESCRIPTION OF THE DRAWINGS

FIG. 1 discloses the hemolyzed blood concentration-time profile of fingolimod following single intravenous bolus administration of fingolimod dose formulation in male Beagle dogs. Semi-log Graph for fingolimod analyte for G1, formulations.

FIG. 2 discloses the blood concentration time profile of fingolimod following intramuscular administration of fingolimod dose formulation in male Beagle dogs; semi-log graph for fingolimod Analyte for G2, G3 & G4 formulations.

### DETAILED DESCRIPTION OF THE INVENTION

The present invention relates to extended release suspension injection compositions consisting essentially of a therapeutically effective amount of fingolimod and an excipient that facilitates intramuscular administration, and methods of use thereof for treating patients with relapsing forms of multiple sclerosis (MS) to reduce the frequency of clinical exacerbations and to delay the accumulation of physical disability.

In embodiments of the present invention further provides the fingolimod extended release suspension injection with sustained release of the fingolimod from the site of injection for about 7 days, 14 days, 21 days, 28 days, 35 days, 42 days, 49 days, 56 days, 63 days, 70 days, 77 days, 84 days, 91 days and 98 days.

In the embodiments of the invention, the invention relates to extended release injectable composition for the extended release of fingolimod comprising fingolimod and a suspending agent. Fingolimod can be present in an amount of at least 0.5 mg/mL, preferably at least about 1 mg/mL, to about 10 mg/mL, and more preferably about 1.5 mg/mL, 2.0 mg/mL, 2.5 mg/mL, 3.5 mg/mL, 4.0 mg/mL, 4.5 mg/mL, 5.0 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL and 10 mg/mL. The invention also relates to methods for providing fingolimod an individual in an extended release injectable composition comprising fingolimod of at least 0.5 mg, more preferably of about 1 mg to about 10 mg, even more preferably of about 1.0 mg, 1.5 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 7.5 mg, 8.0 mg, 8.5 mg, 9.0 mg, 9.5 mg and 10.0 mg and e.g less than 20 mg.

In another embodiment of the invention, the present invention relates to the method of intramuscular administration of fingolimod extended release suspension injection, wherein fingolimod release from the injection site is about 0.05 mg/day to about 1.5 mg/day, preferably from about 0.1 mg/day to about 1.3 mg/day and more preferably from about 0.15 mg/day to about 1.0 mg/day.

In a further embodiment of the invention, the present invention relates to the method of intramuscular administration of fingolimod extended release suspension injection, wherein the mean residence time (MRT) of the fingolimod extended release suspension by intramuscular administration is at least twice the mean residence time (MRT) of fingolimod injection by intravenous administration. The fingolimod extended release suspension injection composition of the present invention increases the mean residence time (MRT) to at least 100 hours, preferably from about 100 hours to about 500 hours, more preferably from about 125 hours to about 350 hours and most preferably from about 150 hours to about 300 hours.

In the most preferred embodiment, the present invention provides the pharmaceutical extended release suspension injection comprises fingolimod and pharmaceutically acceptable excipients.

In embodiments of the invention, the present invention provides the pharmaceutical extended release suspension injectable composition of fingolimod comprising at least about 0.5 mg/mL of fingolimod and a suspending agent and wherein a therapeutically effective amount of fingolimod is present in the plasma of the individual for at least about 7 days.

In an embodiment of the invention, the present invention provides an extended release fingolimod injectable formulation comprising
a) fingolimod having a particle size (D90) of about 5 to 50 microns, and
b) one or more suspending agents,
   wherein said formulation is a homogenous suspension, and wherein upon injection into a subject, said formulation releases fingolimod over a period of at least about 7 days from the date of administration.

In a further embodiment of the invention, the present invention provides and extended release fingolimod injectable formulation comprising
a) fingolimod having a particle size (D90) of about 5 to 50 microns, and
b) one or more suspending agents,
   wherein said formulation is a homogenous suspension, and wherein upon injection into a subject, said formulation releases fingolimod over a period of about 7 days to about 98 days from the date of administration.

Examples of the suspending agent used in the present invention are selected form the group consisting of sodium carboxymethylcellulose, hydroxy propylcellulose, carboxy methylcellulose, hydroxypropyl ethylcellulose, hydroxypropyl methylcellulose, polyvinylpyrrolidone and any combinations thereof. Suspending agent preferably used in the pharmaceutical suspension injection composition of about 1 mg/mL to about 20 mg/mL of the composition i.e in an amount of about 0.1% to about 2% based on the total weight of the composition. Even more preferably, suspending agent is used in the composition of about 5 mg/mL to about 15 mg/mL of the composition i.e about 0.5% to about 1.5% based on total weight of the composition and most preferably is of about 0.9% based on the total weight of the composition. Preferably, the suspending agent is selected from sodium carboxymethylcellulose having a viscosity of about 2 cps to about 1000 cps, more preferably of about 5 cps to about 500 cps, even more preferably of about 7 cps to about 100 cps and most preferably of 8 cps to about 50 cps and even most preferably of about 10 cps to about 45 cps.

In embodiments of the invention the present invention provides an extended release composition comprising a suspension of about 7.0 mg of fingolimod, about 0.9% w/w of suspending agent and water wherein upon administration of the composition the fingolimod release is for at least 7 days.

In embodiments of the invention the present invention provides an extended release composition comprising a suspension of about 7.0 mg of fingolimod, about 0.9% w/w of suspending agent, tonicity adjusting agent and water wherein upon administration of the composition the fingolimod release is for at least 7 days.

In further embodiments of the invention the present invention provides an extended release composition consisting essentially a suspension of about 7.0 mg of fingolimod, about 0.9% w/w of suspending agent, tonicity adjusting agent and water wherein upon administration of the composition the fingolimod release is for at least 7 days.

In embodiments of the invention the composition consists of fingolimod, a suspending agent and water, thereby providing a surprisingly simple and elegant formulation for obtaining an extended or sustained release profile.

The fingolimod drug substance can comprise, consist essentially of or consist of fingolimod (in a crystalline, non-crystalline or amorphous form), a fingolimod salt (fingolimod HCl, fingolimod phosphate), a fingolimod solvate (including hydrates) or other fingolimod polymorphs. The fingolimod or fingolimod drug substance, can be added in a specified size. For example, the fingolimod or fingolimod drug substance can be added after being micronized to D90 of less than about 100 microns, preferably less than about 75 microns, more preferably less than about 60 microns and most preferably between about 5 microns and 50 microns, as determined by master sizer 3000 particle analyzer.

The methods of the invention include administering the compositions described herein, thereby obtaining an extended release or sustained release profile in a patient. An extended release profile that achieve a therapeutically effective amount of fingolimod in plasma after administration in an individual for at least of about 7 days, preferably at least about 14 days, or more preferably at least about 21 days, alternatively for at least 35 days, 42 days, 49 days, 56 days, 63 days, 70 days, 77 days, 84 days, 91 days and 98 days.

In one embodiment, the formulations can be administered as a single or sole dose. However, the invention is particularly beneficial for those individuals that require constant or chronic therapy, such as those that receive repeated doses over several weeks or months or more.

A therapeutically effective amount of the compound used in the treatment described herein can be readily determined by the attending diagnostician, as one skilled in the art, by the use of conventional techniques and by observing results obtained under analogous circumstances. In determining the therapeutically effective dose, a number of factors are considered by the attending diagnostician, including, but not limited to: the species of mammal; it size, age, and general health; the specific disease involved; the degree of or involvement or severity of the disease;

the response of the individual patient; the particular compound administered; the mode of administration; the bioavailability characteristic of the preparation administered; the dosage regimen selected; the use of concomitant medication; and other relevant circumstances.

The mode of administration will generally by injection or implantation, such as intramuscularly or subcutaneously.

In embodiments of the invention, fingolimod injectable suspension of the present invention is administered intramuscularly or subcutaneously as a loading dose or as further dose with the patients being treated for multiple sclerosis with oral fingolimod.

When the composition is to be used as an injectable material, including but not limited to needle-less injection, it can be formulated into a conventional injectable carrier. Suitable carrier includes biocompatible and pharmaceutically acceptable solutions.

In another embodiment, the pharmaceutical extended release suspension injection composition comprising comprising fingolimod, suspending agent, buffer and purified water.

In yet another object, the present invention further provides the extended release suspension injection comprising fingolimod, suspending agent, surfactants, buffering agents, tonicity agents and purified water.

Examples of the surfactants used in the present invention are selected form the group consisting of cetyl pyridinium chloride, gelatin, casein, lecithin (phosphatides), dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens such as e.g., Tween 20 and Tween 80 (ICI Specialty Chemicals)); polyethylene glycol 4000, polysorbate 20, dodecyl trimethyl ammonium bromide, polyoxyethylene stearates, collodial silicon dioxide, phosphates, sodium dodecylsulfate, carboxymethylcellulose calcium, sodium carboxymethyl cellulose, hydroxypropyl celluloses (e.g., HPC, HPC-SL, and HPC-L), methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), 4-(1,1,3,3-tetramethylbutyI)-phenol polymer with ethylene oxide and formaldehyde (also known as tyloxapol, superione, and triton), poloxamers (e.g., Pluronics F68 and F108, which are block copolymers of ethylene oxide and propylene oxide); poloxamines (e.g., Tetronic 908, also known as Poloxamine 908, which is a tetrafunctional block copolymer derived from sequential addition of propylene oxide and ethylene oxide to ethylenediamine (BASF

Wyandotte Corporation, Parsippany, N.J.)) and any combinations thereof. Preferably, the surfactants are selected from polyethylene glycol 4000 and polysorbate 20. Surfactants are preferably used in the pharmaceutical suspension injection composition of about 0.5% to about 15% based on the total weight of the composition. Most preferably, surfactants are used in the composition of about 1% to about 10% based on total weight of the composition.

Examples of the buffering agents are selected from the group consisting of diethanolamine, triethanolamine, sodium hydroxide, phosphate buffer, HEPES, histidine buffer, hydrochloric acid, sodium citrate dihydrate, citric acid and mono basic sodium phosphate. Buffering agent preferably used in the pharmaceutical injection composition of sufficient quantity.

Examples of the tonicity agents are selected from the group consisting of sodium chloride, dextrose, glycerol, mannitol, glucose and thioglycerol. Sodium chloride is the most preferably used tonicity adjusting agent. Sodium chloride is used in an amount of about 5 mg/mL to about 15 mg/mL in the composition.

In embodiments of the invention, the present invention relates to an extended release fingolimod injectable formulation comprising
a) fingolimod having a particle size (D90) of about 5 to 50 microns,
b) sodium carboxymethylcellulose,
c) sodium chloride and
d) water.

In the further specific embodiment, the present invention relates to an extended release fingolimod injectable formulation comprising
a) about 1 mg/mL to about 10 mg/mL of fingolimod having a particle size
(D90) of about 5 to 50 microns,
b) about 5 mg/mL to about 15 mg/mL of sodium carboxymethylcellulose,
c) about 5 mg/mL to about 15 mg/mL of sodium chloride and
d) water.

In a more specific embodiment, the present invention relates to an extended release fingolimod injectable formulation consisting of
a) about 7 mg/mL of fingolimod having a particle size (D90) of about 5 to 50 microns,
b) about 9 mg/mL of sodium carboxymethylcellulose,
c) about 9 mg/mL of sodium chloride and
d) water.

The following example is provided to illustrate the present invention. It is understood, however, that the invention is not limited to the specific conditions or details described in the example below. The example should not be construed as limiting the invention as the examples merely provide specific methodology useful in the understanding and practice of the invention and its various aspects. While certain preferred and alternative embodiments of the invention have been set forth for purposes of disclosing the invention, modification to the disclosed embodiments can occur to those who are skilled in the art.

### Example 1

Suspension injection with the following compositions are prepared.

| S.No | Ingredients | (% w/w) |
|---|---|---|
| 1 | Fingolimod free base | 0.35% |
| 2 | Sodium carboxymethyl cellulose | 0.1%-2% |
| 3 | Purified water Q.S to | 100% |

Process for preparation:
1. Purified Water required for the batch was taken in vessel and purged with nitrogen.
2. 80% of the nitrogen purged water was taken in another vessel and to it Sodium carboxymethyl cellulose was added and dissolved with stirring.
3. Contents of step 2 was sterilization by filtration.
4. Fingolimod free base was added to contents of step 3 and was mixed to form a dispersion of fingolimod.
5. The volume was made up to 100% with remaining purified water.

### Example 2

Suspension injection with the following compositions are prepared.

| S.No | Ingredients | (% w/w) |
|---|---|---|
| 1 | Fingolimod free base | 0.75% |
| 2 | Sodium carboxymethyl cellulose | 0.1%-2% |
| 3 | Purified water Q.S to | 100% |

The process for preparation is similar to process disclosed in example—1.

### Examples 3

Suspension injection with the following compositions are prepared.

| S.No | Ingredients | (% w/w) |
|---|---|---|
| 1 | Fingolimod free base | 0.35% |
| 2 | Sodium carboxymethyl cellulose | 0.1%-2% |
| 3 | Phosphate buffer/histidine buffer | Q.S |
| 4 | Purified water Q.S to | 100% |

Process for Preparation:
1. Purified Water required for the batch was taken in vessel and purged with nitrogen.
2. 90% of the nitrogen purged water was taken in another vessel and the pH was adjusted to in between 5 to 8 with phosphate/histidine buffer.
3. Sodium carboxymethyl cellulose was added to contents of step 2 and dissolved with stirring.
4. Contents of step 3 was sterilization by filtration.
5. Fingolimod free base was added to contents of step 4 and was mixed to form a dispersion of fingolimod.
6. The volume was made up to 100% with remaining purified water.

### Examples 4

Suspension injection with the following compositions are prepared.

| S.No | Ingredients | (% w/w) |
|---|---|---|
| 1 | Fingolimod free base | 0.35% |
| 2 | Sodium carboxymethyl cellulose | 0.1%-2% |
| 3 | Polyethylene glycol 4000 | 0.25%-7.5% |
| 4 | Polysorbate 20 | 0.25%-7.5% |
| 5 | Phosphate buffer/histidine buffer | Q.S |
| 6 | Purified water Q.S to | 100% |

Process for Preparation:
1. The quantity of water for injection required for the batch was collected in a clean and dry glass vessel and subjected for nitrogen purging for 30 minutes to reduce the dissolved oxygen content.
2. About 90% batch size of water for injection from the step 3.1 was collected and histidine/phosphate buffer was prepared [pH 5-8]
3. Dispensed quantity of Sodium CMC was added slowly and stirred till it completely dissolved.
4. Dispensed quantity of Polysorbate 20 was added slowly and stirred till it completely dissolved.
5. Dispensed quantity of Polyethylene glycol 4000 was added slowly and stirred till it completely dissolved
6. The above contents of step 5 are sterilized by filtration.
7. Dispensed quantity of Fingolimod free base was added to the above step.
8. Above suspension was subjected for high shear mixing at 5000-20000RPM for 10-30 minutes.
9. After high shear mixing Fingolimod suspension was subjected for homogenization at 10000-30000 psi for 5-20 cycles to reduce the particle size.
10. Volume of the bulk was made up to 100% batch size with water for injection from and stirred for 10 minutes.

### Example 5

Suspension injection with the following compositions are prepared.

| S.No | Ingredients | mg/ml | (% w/w) |
|---|---|---|---|
| 1 | Fingolimod free base | 3.5 | 0.35 |
| 2 | Sodium carboxymethyl cellulose | 9.0 | 0.9 |
| 3 | Purified water Q.S to | 1 ml | 100% |

The process for preparation is similar to process disclosed in example—1.

### Example 6

Suspension injection with the following compositions are prepared.

| S.No | Ingredients | mg/ml | (% w/w) |
|---|---|---|---|
| 1 | Fingolimod free base | 7.0 | 0.7 |
| 2 | Sodium carboxymethyl cellulose | 9.0 | 0.9 |
| 3 | Sodium Chloride | 9.0 | 0.9 |
| 4 | Purified water Q.S to | 1 ml | 100% |

Process for Preparation:
1. Purified Water required for the batch was taken in vessel and purged with nitrogen.
2. 80% of the nitrogen purged water was taken in another vessel and to it sodium chloride was added and dissolved.
3. After complete solubilization of sodium chloride, Sodium carboxymethyl cellulose was added and dissolved with stirring.
4. Contents of step 3 was sterilization by filtration.
5. Fingolimod free base was added to contents of step 4 and was mixed to form a dispersion of fingolimod.
6. The volume was made up to 100% with remaining purified water.

### Example—7: Pharmacokinetics Parameters of Injectable suspension of

### Example 6 with different particle sizes in comparision to intravenous composition in male beagle dogs.

Male Beagle dogs are divided into three groups as G1, G2, G3 and G4, each consisting of three animals. Fingolimod solution (Formulation 1 is prepared by dissolving 0.5 mg of fingolimod Hcl in 9 mg/mL sodium chloride and up to 1 mL water for injection and pH is adjusted to 3.2 with 0.1N Hydrochloric acid) is administered intravenously (I.V) G1 group and the Injectable suspension composition of example 6 comprising different particle sizes of fingolimod (Formulation 2, 3 and 4) are administered intramuscularly (I.M) to G2, G3 and G4 group animals, with the dose, dose volume and drug concentration as listed in Table —1.

TABLE 1

| | | Particle | | | | |
|---|---|---|---|---|---|---|
| | | size of | | Dose | Drug | |
| | Dose | formulation | Dose | Volume | Conc. | Route of |
| Group | formulation | (D₉₀) | (mg/dog) | (mL/dog) | (mg/mL) | Administration |
| G1 | Formulation 1 | NA | 0.5 | 1 | 0.5 | I.V |
| G2 | Formulation 2 | 5-14μ | 7 | 1 | 7 | I.M |
| G3 | Formulation 3 | 15-25μ | 7 | 1 | 7 | I.M |
| G4 | Formulation 4 | 26μ-50μ | 7 | 1 | 7 | I.M |

Blood samples were collected from each LM route dogs at 0 (pre-dose), 0.25, 0.75, 1.25, 2, 4, 6, 8, 12, 24, 48, 72, 120, 168, 216, 264, 336, 408, 504 hours post-dose. For I.V route dogs, blood samples were collected at 0 (pre-dose), 0.083, 0.167, 0.25, 0.5, 0.75, 1.25, 2, 3, 4, 6, 8, 12, 24, 48, 72, 120, 168, 240 h post-dose. At each time 0 5 mL of blood was withdrawn from jugular vein and transferred to labelled tubes containing equal volume of milli-Q water to obtain hemolyzed blood. The hemolyzed blood samples were analyzed for fingolimod using a fit-for purpose LC-MS/MS method and the pharmacokinetic parameters of fingolimod in hemolyzed blood were calculated using the non-compartmental analysis tool of the validated Phoenix Win Nonlin software (Version 8.0).

The mean residence time (MRT) for the group GI administered with fingolimod intravenously is listed in Table—2 and mean residence time for the group G2, G3 and G4 administered with fingolimod intramuscularly are listed in Table—3.

TABLE 2

| | Route/ | | |
|---|---|---|---|
| | Dose | AUC inf | MRT last |
| Analyte | (mg/Dog) | (ng.h/mL) | (h) |
| Fingolimod | I.V/0.5 | 125 ± 12.0 | 46.4 ± 2.98 |

TABLE 3

| | | | MRT last |
|---|---|---|---|
| Analyte | Group | Route/Dose (mg/Dog) | (h) |
| Fingolimod | G2 | I.M/7 | 166 ± 43.2 |
| | G3 | I.M/7 | 196 ± 10.2 |
| | G4 | I.M/7 | 220 ± 18.1 |

The AUC (h*ng/mL) day-wise split for group 1 with intravenous administration are listed in Table—4 and AUC (h*ng/mL) day wise split for group 2, 3 and 4 with intramuscular administration are listed in Table—5.

TABLE 4

| | Route/ | | | | | |
|---|---|---|---|---|---|---|
| | Dose | | | | Day 14 | Day 21 |
| Group | (mg/Dog) | Day 1 | Day 7 | Day 10 | (Extrapolated) | (Extrapolated) |
| G1 | IV/0.5 | 55.94 | 3.18 | 1.62 | 0.35 | 0.03 |

TABLE 5

| | Route/ | | | | | |
|---|---|---|---|---|---|---|
| | Dose | | | | | |
| Group | (mg/Dog) | Day 1 | Day 7 | Day 10 | Day 14 | Day 21 |
| G2 | IM/7 | 318.83 | 93.38 | 85.32 | 85.67 | 38.60 |
| G3 | IM/7 | 139.18 | 57.04 | 78.36 | 51.05 | 18.08 |
| G4 | IM/7 | 108.26 | 70.72 | 112.72 | 64.15 | 28.25 |

The mg/day exposure in G 1 group animals (intravenous administration) and G2, G3 and G4 group animals are listed in Table—6 and Table—7 respectively.

TABLE 6

| | Route/ | | | | | |
|---|---|---|---|---|---|---|
| | Dose | | | | Day 14 | Day 21 |
| Group | (mg/Dog) | Day 1 | Day 7 | Day 10 | (Extrapolated) | (Extrapolated) |
| G1 | I.V/0.5 | 0.223 | 0.012 | 0.006 | 0.001 | 0.000 |

TABLE 7

| | Route/ | | | | | |
|---|---|---|---|---|---|---|
| | Dose | | | | | |
| Group | (mg/Dog) | Day 1 | Day 7 | Day 10 | Day 14 | Day 21 |
| G2 | I.M/7 | 1.270 | 0.372 | 0.340 | 0.341 | 0.153 |
| G3 | I.M/7 | 0.554 | 0.227 | 0.312 | 0.203 | 0.072 |
| G4 | I.M/7 | 0.431 | 0.281 | 0.449 | 0.255 | 0.112 |

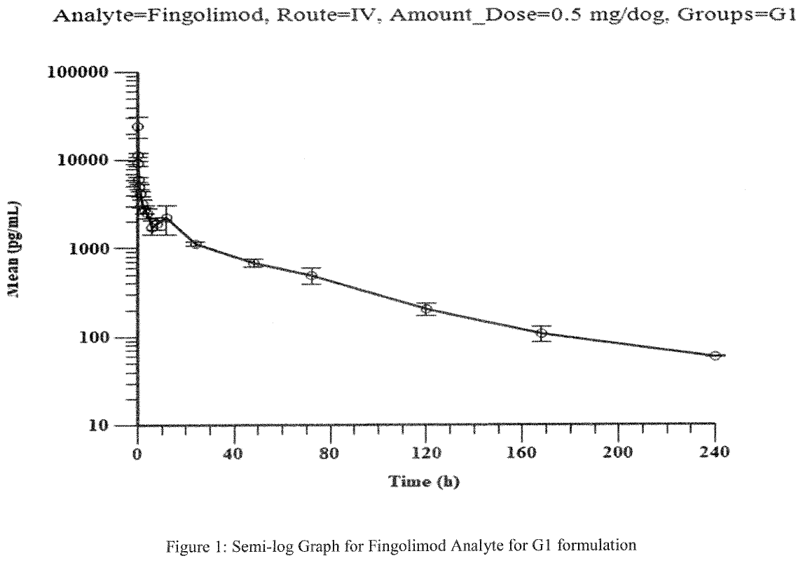

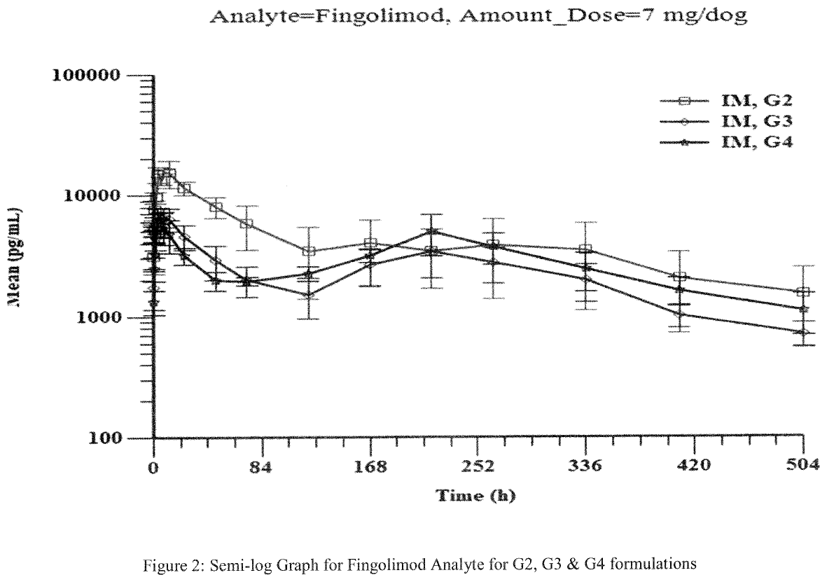

The concentration (pg/mL) vs time points (h) of G1 animal group (intravenous administration) are listed in Table—8 (FIG. 1). The concentration (pg/mL) vs time points (h) of G2, G3 and G4 animal group (intramuscular administration) are listed in Table—9 (FIG. 2).

TABLE 8

| Group/Time | | | | | | | | | | | | |
|---|---|---|---|---|---|---|---|---|---|---|---|---|
| (hour) | 0 | 0.083 | 0.5 | 0.75 | 1.25 | 2 | 4 | 8 | 12 | 24 | 120 | 240 |
| G1 | 0.0 | 24433 | 6023.3 | 4990.0 | 4173.3 | 3243.3 | 2510.0 | 1916.7 | 2233.3 | 1126.7 | 205.00 | 59.400 |
| Mean ± SD | ± | ± | ± | ± | ± | ± | ± | ± | ± | ± | ± | ± |
| pg/mL | 0.0 | 6516 | 448.4 | 329.7 | 285.4 | 320.2 | 347 | 291.4 | 807 | 66.58 | 31.61 | 1.253 |

TABLE 9

| Group/Time | | | | | | | | | | | | |
|---|---|---|---|---|---|---|---|---|---|---|---|---|
| (hour) | 0 | 2 | 4 | 8 | 12 | 24 | 48 | 120 | 264 | 336 | 408 | 504 |
| G2 | 0.0 | 9713.3 | 14900 | 15833 | 15433 | 11577 | 8123.3 | 3443.3 | 3870 | 3509.3 | 2061.7 | 1543.3 |
| Mean ± SD | ± | ± | ± | ± | ± | ± | ± | ± | ± | ± | ± | ± |
| (pg/mL) | 0.0 | 1059 | 2261 | 750.6 | 3782 | 1533 | 1618 | 2052 | 2488 | 2399 | 1333 | 984.8 |
| G3 | 0.0 | 5803.3 | 6683.3 | 6583.3 | 6416.7 | 4576.7 | 2920 | 1507 | 2780 | 1996.7 | 1020.3 | 715 |
| Mean ± SD | ± | ± | ± | ± | ± | ± | ± | ± | ± | ± | ± | ± |
| (pg/mL) | 0.0 | 780.5 | 1251 | 1139 | 1364 | 1047 | 920.7 | 546.7 | 922.6 | 684.1 | 224.1 | 165.3 |
| G4 | 0.0 | 4360 | 6310 | 5640 | 4780 | 3176.7 | 1993.3 | 2256.7 | 3736.7 | 2460 | 1626.7 | 1113 |
| Mean ± SD | ± | ± | ± | ± | ± | ± | ± | ± | ± | ± | ± | ± |
| (pg/mL) | 0.0 | 831.6 | 1490 | 1755 | 1475 | 518.7 | 357 | 315.3 | 1065 | 854.6 | 425.2 | 414.2 |

We claim:

1. An extended release fingolimod injectable formulation comprising
a) fingolimod having a particle size (D90) of about 5 to 50 microns, and
b) one or more suspending agents,
wherein said formulation is a homogenous suspension, and wherein upon injection into a subject, said formulation releases fingolimod over a period of at least about 7 days from the date of administration.

2. The extended release fingolimod injectable formulation as claimed in claim 1, wherein the formulation comprises about 1 mg/mL to about 10 mg/mL of fingolimod.

3. The extended release fingolimod injectable formulation as claimed in claim 1, wherein the formulation comprises about 5 mg/mL to about 15 mg/mL of suspending agent.

4. The extended release fingolimod injectable formulation as claimed in claim 3, wherein suspending agents are selected from group consisting of sodium carboxymethylcellulose, hydroxypropyl cellulose, carboxymethyl cellulose, hydroxypropyl ethylcellulose, hydroxypropyl methylcellulose and polyvinylpyrrolidone.

5. The extended release fingolimod injectable formulation as claimed in claim 1, wherein said formulation further comprises tonicity adjusting agent and purified water.

6. The extended release fingolimod injectable formulation as claimed in claim 5, wherein said formulation comprises
a) fingolimod having a particle size (D90) of about 5 to 50 microns,
b) sodium carboxymethylcellulose,
c) sodium chloride and
d) water.

7. The extended release fingolimod injectable formulation as claimed in claim 6, wherein the formulation comprises
a) about 1 mg/mL to about 10 mg/mL of fingolimod having a particle size (D90) of about 5 to 50 microns,
b) about 5 mg/mL to about 15 mg/mL of sodium carboxymethylcellulose,
c) about 5 mg/mL to about 15 mg/mL of sodium chloride and
d) water.

8. A fingolimod extended release suspension comprising
a) fingolimod having a particle size (D₉₀) of about 5 to 50 microns, and
b) one or more suspending agents,
wherein the mean residence time (MRT) of the fingolimod extended release suspension by intramuscular administration is at least twice the mean residence time (MRT) of fingolimod injection by intravenous administration.

9. The fingolimod extended release suspension as claimed in claim 8, wherein the suspension comprises
a) fingolimod having a particle size (D90) of about 5 to 50 microns,
b) sodium carboxymethylcellulose,
c) sodium chloride and
d) water.

10. The fingolimod extended release suspension as claimed in claim 9, wherein the suspension comprises
a) about 1 mg/mL to about 10 mg/mL of fingolimod having a particle size (D₉₀) of about 5 to 50 microns,
b) about 5 mg/mL to about 15 mg/mL of sodium carboxymethylcellulose,
c) about 5 mg/mL to about 15 mg/mL of sodium chloride and
d) water.
